(19) United States
(12) Patent Application Publication
(10) Pub. No.: US 2023/0118225 A1
(43) Pub. Date: Apr. 20, 2023

(54) MOVEABLE IONIZATION UNIT FOR CLEANING AIR IN A ROOM WITH A SUPPORT STRUCTURE

(71) Applicant: PHILIP MORRIS PRODUCTS S.A., Neuchatel (CH)

(72) Inventors: Rui Nuno Batista, Morges (CH); Ricardo Cali, Mannheim (DE); Louis Beck, Lausanne (CH)

(21) Appl. No.: 17/911,204

(22) PCT Filed: Mar. 16, 2021
(86) PCT No.: PCT/EP2021/056636

§ 371 Date: Sep. 13, 2022

(30) Foreign Application Priority Data
Mar. 16, 2020 (EP) 20163327.8

(51) Int. Cl. F24F 8/30; B03C 3/017; B03C 3/32; B03C 3/12; B03C 3/38

(52) U.S. Cl. CPC F24F 8/30; B03C 3/017; B03C 3/32; B03C 3/12; B03C 3/38; F24F 2221/42; F24F 2221/14; H01T 23/00

(57) ABSTRACT

A Method for cleaning air in a room (1) with a ceiling (3) and a floor (5), comprising moving an ionization unit (13) above the floor (5) along a support structure provided at a distance to the floor (5); and electrically charging particles in the air by the ionization unit (13).

The present disclosure relates to cleaning air in a room. According to embodiments, the present disclosure relates to cleaning air in a cleanroom.

U.S. Pat. No. 5,626,820 A describes an air handling system for introducing air into a clean room. The air handling system comprises a combination of a HEPA filter (high-efficiency particulate air filter) and a chemical filter. The floor of the clean room has air passages to allow air streams to pass through. There is an air stream recirculation rate in the order of 10 interchanges per minute.

Ionic air purifiers for home use are known from practice. Those devices comprise stationary ionizers creating negative ions in the air.

DE 10 2004 036 459 A1 discloses a self-driving disc-shaped cleaning robot having a vacuum unit for sucking in dust from a floor. The cleaning robot further comprises a negative ion generation unit provided in the robot body to carry out an air cleaning operation.

According to an aspect of the present invention, there is provided a method for cleaning air in a room with a ceiling and floor. The method comprises moving an ionization unit above the floor along a support structure provided at a distance of the floor. The method further comprises electrically charging particles in the air by the ionization unit.

As the ionization unit moves above the floor along a support structure provided at a distance to the floor, the ionization unit may electrically charge particles in the air at a distance to the floor. Electrically charged particles in the air may attract other particles in the air due to electrostatic interaction. Electrically charging particles in the air by the ionization unit may lead to the formation of clusters of particles due to electrostatic interaction. Formation of clusters of particles at a distance to the floor may increase the tendency of the particles to descend due to gravity. Clusters of particles within the air may descend at a faster velocity than individual particles. Electrically charging particles at a distance to the floor may contribute to removing particles from the air by increasing their tendency to settle down on the floor or another surface. As the ionization unit is moving, the ionization unit may be able to treat different regions within the room at different times. As the ionization unit moves along the support structure provided at a distance to the floor, the ionization unit may operate essentially without disturbing activities carried out below the ionization unit.

The room may comprise one or more walls. The one or more walls of the room may connect the floor and the ceiling. The room may comprise a space delimited by the floor, the one or more walls and the ceiling. The ceiling may comprise an intermediate ceiling. The ceiling may be a structural part of a gallery. The floor may be at least substantially horizontal. The ceiling may be at least substantially horizontal. The room may be part of a building.

The room may comprise a space in which a person may move, in particular by walking around. The space in which the person may move may be delimited in a downward direction by the floor. A person within the space may stand on the floor or walk on the floor. The space in which the person may move may be delimited in an upward direction by the ceiling. A person within the space may be able to touch the ceiling (provided he is tall enough or uses a ladder or the like). The space in which the person may move may be delimited in lateral directions by one or more walls of the room. The one or more walls of the room may connect the floor and the ceiling.

The particles may comprise dust particles or other particulate matter. The particles may comprise particles floating in the air.

The method may comprise supplying air through the ceiling of the room. The air may be supplied through the ceiling of the room in a downward direction. The downward direction may be a vertical direction or an essentially vertical direction. As the ionization unit moves along the support structure provided at a distance to the floor, air supplied through the ceiling of the room may be treated by the ionization unit after having been supplied through the ceiling of the room.

The method may comprise discharging air through the floor of the room. The air may be discharged through the floor of the room in a downward direction. The downward direction may be a vertical direction or an essentially vertical direction. Airflow through the floor of the room may carry clusters of particles created due to operation of the ionization unit and may discharge those clusters of particles together with the air.

One or both of the ceiling and the floor may be at least partially air-permeable. One or more air channels may be provided through the ceiling. One or more air channels may be provided through the floor. A plurality of air channels going through the ceiling may be distributed over the ceiling. A plurality of air channels going through the floor may be distributed over the floor. The air channels may extend vertically or essentially vertically.

The method may comprise circulating air from a circulation space of the room through the ceiling of the room and further through the floor of the room into the circulation space. A permanent flow of air from the ceiling to the floor may be generated. Air flowing from the ceiling to the floor may carry clusters of particles towards the floor.

The air may be filtered within the circulation space. Filtering may be carried out by HEPA filters (high-efficiency particulate air filters), for example.

The ionization unit may charge the particles in the air by way of corona discharge. The ionization unit may use the Piezoelectric Direct Discharge Effect to generate ions. The ionization unit may generate cold plasma. The ionization unit may, for example, comprise a piezo transformer, in particular a Rosen-type piezo transformer. The piezo transformer may have a resonance frequency between 10 kHz and 500 kHz, preferably between 200 kHz and 300 kHz.

The ionization unit may move at the ceiling of the room. The ceiling of the room may constitute the support structure. The ceiling of the room may comprise the support structure. If the ionization unit moves at the ceiling of the room, disturbance of activities within the room due to operation of the ionization unit may be minimized. Operation of the ionization unit may cause particles at or near the ceiling to form clusters and descend within the room.

The ionization unit may hang from the ceiling while moving at the ceiling. Hanging from the ceiling may comprise being supported at the ceiling while being positioned below the ceiling.

The ionization unit may be held at the support structure with suction cups provided at the ionization unit. The ionization unit may be suspended from the support structure with suction cups provided at the ionization unit. In particular, the ionization unit may be held at the ceiling or be suspended from the ceiling with suction cups provided at the ionization unit. If suction cups are provided at the ionization unit, it is not required to provide the support structure with elaborate means for holding the ionization unit. The support structure may comprise a flat surface for engagement with the suction cups. The flat surface may be an extensive flat surface or the flat surface may form one or more paths along which the ionization unit moves. The suction cups may be of various shapes and forms. The suction cups may be generally circular or elliptical or may have irregular shapes. The suction cups may be of various dimensions. For example, the suction cups may have diameters between 5 mm and 80 mm, or between 10 mm and 50 mm, or between 10 mm and 40 mm. The suction cups may also be of a smaller dimension and may have smaller diameters, such as diameters between 0.001 mm and 5 mm, or between 0.001 mm and 1 mm, or between 0.001 mm and 0.1 mm. The suction cups may be part of a micro-suction-surface. The ionization unit may comprise a suction unit selectively creating an underpressure between one or more suction cups and the support structure. However, the suction cups may also be functional to hold the ionization unit at the support structure without a suction unit.

The suction cups of the ionization unit may be provided at a rotatable structure of the ionization unit for moving the ionization unit. For example, the suction cups may be provided at a caterpillar device of the ionization unit or at wheels of the ionization unit.

The support structure may comprise a rail system. A rail system allows predefining the path of movement of the ionization unit. The ionization unit may move along the rail system. The rail system may be provided at the ceiling of the room. The ionization unit may be suspended from the rail system.

The ionization unit may be powered through the rail system. Powering the ionization unit through the rail system removes the need for providing the ionization unit with its own power source.

The ionization unit may move on top of the support structure. In particular, the ionization unit may move on top of the ceiling. If the ionization unit moves on top of the support structure, a drive system of the ionization unit may be comparatively simple. For example, the ionization unit may be provided with wheels for driving on the support structure. The ionization unit may drive freely on the support structure. Alternatively, a predetermined path for the ionization unit may be defined on the support structure, for example by proving rails or guide channels.

The ionization unit may move at a distance of at least 50 centimeters, or at least 80 centimeters, or at least 100 centimeters, or at least 150 centimeters, or at least 200 centimeters, or at least 250 centimeters above the floor of the room. The ionization unit may move at a distance of less than 10 meters, or less than 8 meters, or less than 6 meters, or less than 4 meters, or less than 3 meters above the floor.

A distance between the floor and the support structure may be least 50 centimeters, or at least 80 centimeters, or at least 100 centimeters, or at least 150 centimeters, or at least 200 centimeters, or at least 250 centimeters. A distance between the floor and the support structure may be less than 10 meters, or less than 8 meters, or less than 6 meters, or less than 4 meters, or less than 3 meters.

The ionization unit may continuously move along the support structure. Alternatively, the ionization unit may move intermittently along the support structure.

The ionization unit may move along the support structure at a velocity between 1 centimeters/minute and 100 centimeters/minute, or at a velocity between 5 centimeters/minute and 70 centimeters/minute, or at a velocity between 5 centimeters/minute and 50 centimeters/minute.

The method may further comprise moving a cleaning robot at the floor of the room in coordination with the movement of the ionization unit. Coordination between the ionization unit and the cleaning robot may allow the ionization unit and the cleaning robot to work together to improve cleaning efficiency. The cleaning robot may be controlled based on movement of the ionization unit.

The cleaning robot may continuously move at the floor. Alternatively, the cleaning robot may move intermittently at the floor.

The ionization unit and the cleaning robot may both move intermittently. The ionization unit and the cleaning robot may both move continuously. The ionization unit may move intermittently and the cleaning robot may move continuously. The ionization unit may move continuously and the cleaning robot may move intermittently.

The ionization unit and the cleaning robot may move so as to be positioned above each other. According to an embodiment, the ionization unit and the cleaning robot may move directly above each other so that there is at least one point on the ionization unit that vertically overlaps with a point on the cleaning robot. Alternatively, the ionization unit and the cleaning robot may be positioned above each other within a certain tolerance. For example, the cleaning robot may move on the floor within a region around a vertical projection of the ionization unit onto the floor. A distance between the cleaning robot and the vertical projection of the ionization unit onto the floor may, for example, be kept lower than 5 m, or lower than 3 m, or lower than 2 m, or lower than 1 m, or lower than 0.5 m, or lower than 0.2 m, or lower than 0.1 m.

One of the ionization unit and the cleaning robot may be configured as lead unit and the other one of the ionization unit and the cleaning robot may be configured as follow unit. The follow unit may move according to movements of the lead unit. A movement pattern of the follow unit may be determined based on movements of the lead unit. There may be a time delay between movement of the lead unit and movement of the follow unit. The time delay may be at least 1 second, or at least 3 seconds, or at least 5 seconds, or at least 20 seconds, or at least 40 seconds, or at least 60 seconds, or at least 120 seconds. The time delay may be lower than 500 seconds, or lower than 300 seconds, or lower than 150 seconds, or lower than 80 seconds, or lower than 30 seconds, or lower than 10 seconds. The time delay may correspond to a period of time that passes between the lead unit leaving a first spatial area and the follow unit occupying a second spatial area, the vertical projection of the second special area overlapping with the first spatial area. The cleaning robot may be configured to follow a movement of the ionization unit. The ionization unit may be configured to follow a movement of the cleaning robot.

The cleaning robot may attract charged particles in the air with at least one electrically charged surface. The electrically charged surface may interact with charged particles in the air to draw the charged particles towards the cleaning robot.

The cleaning robot may suck in air and discharge the air in a downward direction. By sucking in air from within the room, the cleaning robot may suck in particles comprised in the air. Discharging the air in a downward direction may be useful to prevent horizontal airflow within the room so as to avoid spreading particles within the room.

The cleaning robot may suck in the air through an inlet opening facing towards an upside direction. If the inlet opening faces towards an upside direction, the cleaning robot may suck in particles falling down within the room due to gravity.

The cleaning robot may move on top of the floor. If the cleaning robot moves on top of the floor, the cleaning robot may suck in particles falling down within the room before the particles reach the floor.

The cleaning robot may move below the floor. A cleaning robot moving below the floor may cause less disturbance to activities carried out on the floor. If the cleaning robot moves below the floor, air movements caused by operation of the cleaning robot may be less likely to spread particles in a space above the floor.

According to another aspect of the present invention, there is provided a system comprising a room and an ionization unit. The room comprises a floor and ceiling. The room comprises a support structure provided at a distance to the floor. The ionization unit is configured to move above the floor along the support structure and electrically charge particle within the room.

The ceiling may comprise the support structure.

The ionization unit may comprise suction cups configured to hold the ionization unit at the support structure.

The support structure may comprise a rail system.

The rail system may be configured to power the ionization unit.

The ceiling and the floor may be at least partially air-permeable.

The room may further comprise a circulation space and circulation system. The circulation system may be configured to circulate air from the circulation space through the ceiling of the room and further through the floor of the room into the circulation space. The circulation space may be in fluid communication with the room.

The system may further comprise a filter unit configured to filter the air within the circulation space.

The ionization unit may be configured to charge the particles in the air by way of corona discharge.

The system may further comprise a cleaning robot configured to move in coordination with the movement of the ionization unit.

The cleaning robot may be configured to move at the floor of the room.

The cleaning robot may comprise at least one electrically charged surface configured to attract charged particles.

The room may be configured as a cleanroom. The room may be configured as a cleanroom of class 10 or lower, or as a cleanroom of class 9 or lower, or as a cleanroom of class 8 or lower, or as a cleanroom of class 7 or lower, or as a cleanroom of class 6 or lower, or as a cleanroom of class 5 or lower, or as a cleanroom of class 4 or lower, wherein the classes are as defined in DIN EN ISO 14644.

According to another aspect of the present invention, there is provided a use of an ionization unit moving within a room to accelerate gravitation-based descent of particles within the room.

Use of an ionization unit allows to accelerate gravitation-based descent of particles without creating larger air movements and disturbances within the room.

The particles may comprise dust particles or other particulate matter. The particles may comprise particles floating in the air.

The ionization unit may move in the room along a support structure provided at a distance to a floor of the room.

The ionization unit may move at height of at least 50 centimeters, or at least 80 centimeters, or at least 100 centimeters, or at least 150 centimeters, or at least 200 centimeters, or at least 250 centimeters above a floor of the room. A particle that is charged by the ionization unit high above the floor may have plenty opportunities to electrostatically attract other particles floating in the air on its way towards the floor.

The ionization unit may electrically charge dust particles in the air to cause the particles to form clusters. Clusters of dust

According to another aspect of the present invention, there is provided a cleaning robot comprising a robot body, a drive unit and an airflow unit. The robot body has an air inlet and an air outlet. The drive unit is configured to move the cleaning robot over a ground surface. The airflow unit is configured to suck outside air into the robot body through the air inlet and to discharge the air from the robot body through the air outlet. The air outlet is positioned at the robot body such that the air discharged through the air outlet is directed towards the ground surface.

The ground surface may be a floor of a room or a surface below the floor of a room, for example.

Having the air outlet positioned at the robot body such that the air discharged through the air outlet is directed towards the ground surface reduces distribution of dust above the ground surface as compared to an air outlet discharging air towards lateral sides or an upper side of the cleaning robot. The cleaning robot is particularly suitable for use on an air-permeable ground surface, as air discharged through the air outlet may pass through the ground surface to remove particles within the air from the space above the ground surface.

The air inlet may open towards the outside of the robot body in a direction facing away from the ground surface. The air inlet opening in a direction facing away from the ground surface facilitates sucking in dust floating in the air provided in the space above the ground surface. The air inlet may open towards the outside of the robot body in an upward direction.

The cleaning robot may further comprise an air passage connecting the air inlet with the air outlet within the robot body. The air passage may be configured to let air introduced through the air inlet flow to the air outlet and be discharged through the air outlet in a filtration-free manner. Discharging the air in a filtration-free manner removes the need of maintenance or replacement of a filter. Air filtration may instead be carried out by an external filtration system, which may be part of an air circulation system, for example.

The cleaning robot may comprise at least one electrically charged surface configured to attract charged particles floating in the air. The electrically charged surface may cause electrically charged particles floating in the air to come within a suction range of the cleaning robot.

The at least one electrically charged surface may comprise an air-permeable conductor provided at the air inlet.

Different aspects of the present invention provide a method, a system, a use and cleaning robot. Any one or more of the features of these aspects may be combined with any one or more features of all other aspects.

Below, there is provided a non-exhaustive list of non-limiting examples, embodiments, or aspects of the invention. Any one or more of the features of these examples, embodiments, or aspects of the invention may be combined with any one or more features of another example, embodiment or aspect described herein.

Example A1: Method for cleaning air in a room with a ceiling and a floor, comprising:

moving an ionization unit above the floor along a support structure provided at a distance to the floor; and

electrically charging particles in the air by the ionization unit.

Example A2: Method according to example A1, further comprising supplying air through the ceiling of the room.

Example A3: Method according to example A1 or A2, further comprising discharging air through the floor of the room

Example A4: Method according to any one of examples A1 to A3, wherein the ceiling and the floor are at least partially air-permeable.

Example A5 Method according to any one of examples A1 to A4, further comprising circulating air from an circulation space of the room through the ceiling of the room and further through the floor of the room into the circulation space, wherein the ceiling and the floor are at least partially air-permeable.

Example A6: Method according to example A5, further comprising filtering the air within the circulation space.

Example A7: Method according to any one of examples A1 to A6, wherein the ionization unit charges the particles in the air by way of corona discharge.

Example A8: Method according to any one of examples A1 to A6, further comprising moving a cleaning robot at the floor of the room in coordination with the movement of the ionization unit.

Example A9: Method according to example 8, wherein the ionization unit and the cleaning robot move so as to be positioned above each other.

Example A10: Method according to example A8 or A9, wherein the cleaning robot attracts charged particles in the air with at least one electrically charged surface.

Example A11: Method according to any one of examples A8 to A10, wherein the cleaning robot sucks in air and discharges the air in a downward direction.

Example A12: Method according to example A11, wherein the cleaning robot sucks in the air through an inlet opening facing towards an upside direction.

Example A13: Method according to any one of examples A8 to A12, wherein the cleaning robot moves on top of the floor.

Example A14: Method according to any one of examples A8 to A12, wherein the cleaning robot moves below the floor.

Example A15: Method according to any one of examples A1 to A14, wherein the ionization unit moves at the ceiling of the room.

Example A16: Method according to any one of examples A1 to A15, wherein the ionization unit hangs from the ceiling while moving at the ceiling.

Example A17: Method according to any one of examples A1 to A16, wherein the ionization unit is held at the support structure with suction cups provided at the ionization unit.

Example A18: Method according to example A17, wherein the suction cups are provided at a rotatable structure of the ionization unit for moving the ionization unit.

Example A19: Method according to any one of examples A1 to A16, wherein the support structure comprises a rail system.

Example A20: Method according to example A19, wherein the ionization unit is powered through the rail system.

Example A21: Method according to any one of examples A1 to A15, wherein the ionization unit moves on top of the support structure.

Example B1: System, comprising:

a room with a floor and a ceiling, wherein the room comprises a support structure provided at a distance to the floor; and

an ionization unit configured to move above the floor along the support structure and electrically charge particles within the room.

Example B2: System according to example B1, wherein the ceiling comprises the support structure.

Example B3: System according to example B1 or B2, wherein the ionization unit comprises suction cups configured to hold the ionization unit at the support structure.

Example B4: System according to any one of examples B1 to B3, wherein the support structure comprises a rail system.

Example B5: System according to example B4, wherein the rail system is configured to power the ionization unit.

Example B6: System according to any one of examples B1 to B5, wherein the ceiling and the floor are at least partially air-permeable.

Example B7: System according to any one of examples B1 to B6, wherein the room further comprises a circulation space and a circulation system configured to circulate air from the circulation space through the ceiling of the room and further through the floor of the room into the circulation space.

Example B8: System according to example B7, further comprising a filter unit configured to filter the air within the circulation space.

Example B9: System according to any one of examples B1 to B8, wherein the ionization unit is configured to charge the particles in the air by way of corona discharge.

Example B10: System according to any one of examples B1 to B9, further comprising a cleaning robot configured to move in coordination with the movement of the ionization unit.

Example B11: System according to example B10, wherein the cleaning robot is configured to move at the floor of the room.

Example B12: System according to example B10 or B11, wherein the cleaning robot comprises at least one electrically charged surface configured to attract charged particles.

Example C1: Use of an ionization unit moving within a room to accelerate gravitation-based descent of particles within the room.

Example C2: Use according to example C1, wherein the ionization unit moves at a height of at least 50 centimeters or at least 80 centimeters or at least 100 centimeters or at least 150 centimeters or at least 200 centimeters or at least 250 centimeters above a floor of the room.

Example C3: Use according to example C1 or C2, wherein the ionization unit electrically charges particles in the air to cause the particles to form clusters.

Example D1: Cleaning robot, comprising:

a robot body having an air inlet and an air outlet;

a drive unit configured to move the cleaning robot over a ground surface; and

an airflow unit configured to suck outside air into the robot body through the air inlet and to discharge the air from the robot body through the air outlet,

wherein the air outlet is positioned at the robot body such that the air discharged through the air outlet is directed towards the ground surface.

Example D2: Cleaning robot according to example D1, wherein the air inlet opens towards the outside of the robot body in a direction facing away from the ground surface.

Example D3: Cleaning robot according to example D1 or D2, further comprising an air passage connecting the air inlet with the air outlet within the robot body, wherein the air passage is configured to let air introduced through the air inlet flow to the air outlet and be discharged through the air outlet in a filtration-free manner.

Example D4: Cleaning robot according to any one of examples D1 to D3, further comprising at least one electrically charged surface configured to attract charged particles floating in the air.

Example D5: Cleaning robot according to example D5, wherein the at least one electrically charged surface comprises an air-permeable conductor provided at the air inlet.

Examples will now be further described with reference to the figures in which:

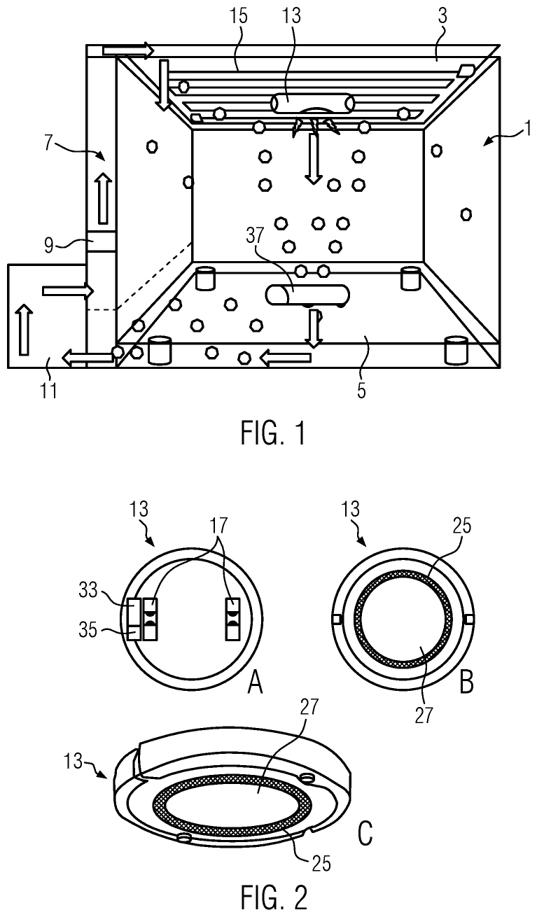

FIG. 1 shows a schematic view of a room with an ionization unit moving along rails at the ceiling of the room according to an embodiment of the invention;

FIG. 2 shows schematic top, bottom and perspective views of the ionization unit of FIG. 1;

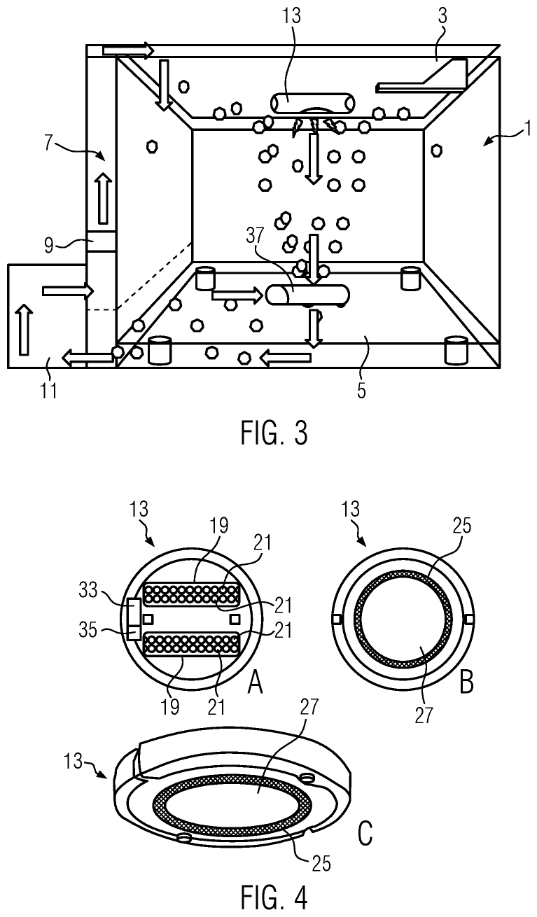

FIG. 3 shows a schematic view of a room with an ionization unit moving along the ceiling of the room according to an embodiment of the invention;

FIG. 4 shows schematic top, bottom and perspective views of the ionization unit of FIG. 3;

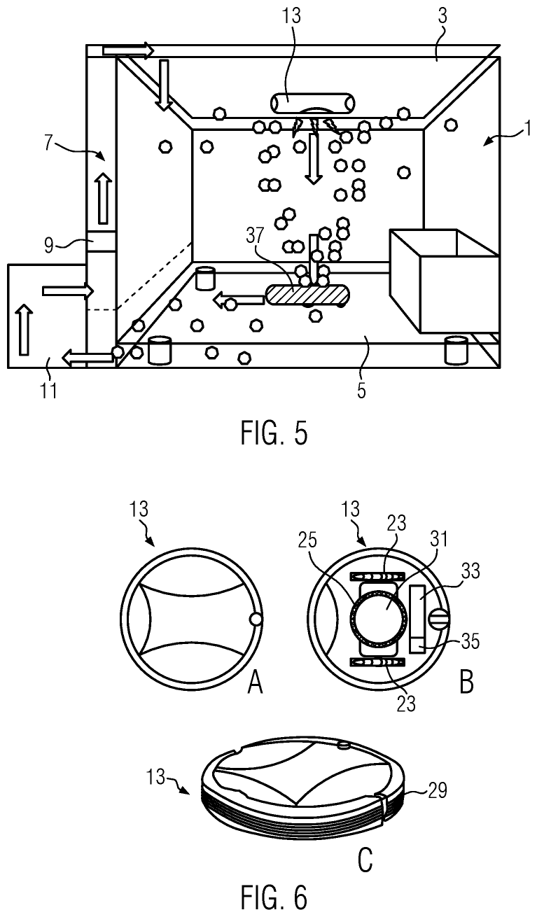

FIG. 5 shows a schematic view of a room with an ionization unit moving on top of the ceiling of the room according to an embodiment of the invention;

FIG. 6 shows schematic top, bottom and perspective views of the ionization unit of FIG. 5;

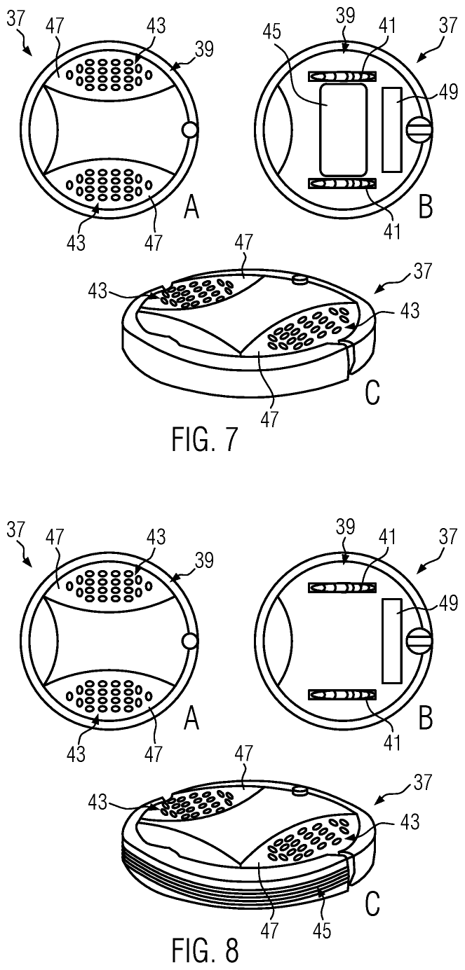

FIG. 7 shows schematic top, bottom and perspective views of a cleaning robot moving on the floor in FIGS. 1 and 3; and

FIG. 8 shows schematic top, bottom and perspective views of a cleaning robot moving below the floor in FIG. 5.

Aspects of the invention relate to cleaning air in a room 1. As shown in FIGS. 1, 3 and 5, the room 1 comprises a ceiling 3 and a floor 5. In the illustrated embodiments, the room 1 is a cleanroom. However, the invention could also be applied to other kinds of rooms. In the cleanroom embodiments illustrated in FIGS. 1, 3 and 5, the room 1 comprises a circulation space 7 comprising a space above the ceiling 3, a space below the floor 5 and a space connecting the space above the ceiling 3 and the space below the floor 5 with each other. The ceiling 3 and the floor 5 of the room 1 are air-permeable. For example, the ceiling 3 and the floor 5 may comprise air channels allowing air to pass through the ceiling 3 and the floor 5 in a vertical direction. A circulation system 9, is provided to continuously circulate air from the circulation space 7 through the ceiling 3 (in a downward direction), through the floor 5 (in a downward direction) into the circulation space 7 and again through the ceiling 3 (in a downward direction). Due to the circulation system 9 circulating the air, there will be a continuous flow of air within the room 1 from the ceiling 3 towards the floor 5. A filter 11 is provided within the circulation space 9 to purify the air during circulation. The filter 11 may be configured to remove particles, such as dust particles or other particles, from the air. In the embodiments of FIGS. 1, 3 and 5, an ionization unit 13 moves along the ceiling 3.

The ionization unit 13 may continuously move along the ceiling 3. For example, the ionization unit 13 may continuously move along the ceiling 3 at a velocity between 1 centimeters/minute and 100 centimeters/minute, or at a velocity between 5 centimeters/minute and 70 centimeters/minute, or at a velocity between 5 centimeters/minute and 50 centimeters/minute. Alternatively, the ionization unit 13 may move intermittently along the ceiling 3. For example, the ionization unit 13 may be controlled to remain at its present location until a particle density in the air detected by a particle sensor is below a predetermined threshold. If the detected particle density is below the predetermined threshold, the ionization unit 13 may move to another position. Alternatively, the ionization unit 13 may remain at one location for a predetermined time and move to the next location after the predetermined time has expired.

The way in which the ionization unit 13 moves along the ceiling 3 is different in the embodiments of FIGS. 1, 3 and 5.

In the embodiment of FIG. 1, the ionization unit 13 hangs from the ceiling 3 and moves along a rail system 15 provided at the ceiling 3. The rail system 15 comprises rails defining a continuous path at the ceiling 3 along which the ionization unit 13 travels. According to a preferred embodiment, the ionization unit 13 is powered via the rail system 15. Alternatively, the ionization unit 13 could comprise a rechargeable power source, such as a rechargeable battery, or could be connected to a power supply by a wire. FIG. 2 shows details of the ionization unit 13 of FIG. 1. Part A of FIG. 2 shows a top view of the ionization unit 13 of FIG. 1, Part B or FIG. 2 shows a bottom view of the ionization unit 13 of FIG. 1 and Part C of FIG. 2 shows a perspective bottom view of the ionization unit 13 of FIG. 1. As shown in Part A of FIG. 2, the ionization unit 13 comprises a rail drive 17 for engaging the rail system 15. The rail drive 17 may cooperate with the rail system 15 to hold the ionization unit 13 at the ceiling 3 and to allow the ionization unit 13 to move along the rail system 15.

According to the embodiment of FIG. 3, the ionization unit 13 moves along the ceiling 3 without relying on a rail system. The ionization unit 13 hangs from the ceiling 3 and freely moves along the ceiling 3 (without being restricted to particular rails or guide structures provided at the ceiling 3). FIG. 4 illustrates details of the ionization unit 13 of FIG. 3. Part A of FIG. 4 shows a top view of the ionization unit 13 of FIG. 3. Part B of FIG. 4 shows a bottom view of the ionization unit 13 of FIG. 3 and Part C of FIG. 4 shows a bottom perspective view of the ionization unit 13 of FIG. 3. As shown in Part A of FIG. 4, the ionization unit 13 comprises a rotatable structure 19 for engaging the ceiling 3. In the illustrated case, there are two rotatable structures 19 and the rotatable structures 19 are embodied as caterpillar devices. The ionization unit 13 comprises a drive unit for driving the rotatable structures 19 to move the ionization unit 13 along the ceiling 3. To hold the ionization unit 13 at the ceiling 3, suction cups 21 are provided at the rotatable structures 19. The ionization unit 13 comprises a suction device configured to apply underpressure between the suction cups 21 and the ceiling 3 to generate a holding force for holding the ionization unit 13 at the ceiling 3. The ionization unit 13 may comprise a rechargeable power source, such as a rechargeable battery. The ionization unit 13 might also be powered via a wired connection.

In the embodiment shown in FIG. 5, the ionization unit 13 moves along the ceiling 3 on top of the ceiling 3. As in FIG. 3, the ionization unit 13 of FIG. 5 moves freely along the ceiling 3 and is not limited by guide structures at the ceiling 3, such as rails. FIG. 6 shows details of the ionization unit 13 of FIG. 5. Part A of FIG. 6 shows a top view of the ionization unit 13 FIG. 5, Part B of FIG. 6 shows a bottom view of the ionization unit 13 FIG. 5 and Part C of FIG. 6 shows a top perspective view of the ionization unit 13 FIG. 5. As showing in Part B of FIG. 6, the ionization unit 13 comprises wheels 23 for engaging a top surface of the ceiling 3 and enabling the ionization unit 13 to travel on top of the ceiling 3 along the ceiling 3. The ionization unit 13 comprises a drive unit for driving the wheels 23. The ionization unit 13 may comprise a rechargeable power source, such as a rechargeable battery. The ionization unit 13 might also be powered via a wired connection.

The ionization unit 13 (according the embodiments of FIGS. 1, 3 and 5) comprises an ionizer 25 for electrically charging particles in the air. The ionizer 25 may comprise a piezo transformer, in particular a Rosen-type piezo transformer. The ionizer 25 may charge particles in the air by creating a corona discharge. In particular, the ionizer 25 may negatively charge particles in the air. Alternatively, the ionizer 25 might positively charge particles in the air. Particles charged by the ionizer 25 tend to form clusters with other particles in the air. Such clusters of particles descend within the room 1 at an increased rate. Clusters of particles may settle down in the room 1 faster than individual particles.

As illustrated in FIGS. 2, 4 and 6, the ionizer 25 may face towards the floor 3. The ionizer 25 illustrated in FIGS. 2, 4 and 6 is ring-shaped. However, any suitable shapes of the ionizer 25 are conceivable.

Parts of the ionization unit 13 may be air-permeable. Air-permeable sections at the ionization unit 13 may allow air to pass through the ionization unit 13 to not shut off the airflow from the ceiling 3 to floor 5 at the position of the ionization unit 13. For example, in the embodiments of FIGS. 2 and 4, a central portion of the ionization unit 13 that is surrounded by the ionizer 25 at the lower side of the ionization unit 13 may comprise a through channel 27 for allowing air to pass through the ionization unit 13. The ionization units 13 illustrated in FIGS. 2 and 4 may have portions that allow flow of air through the ionization unit 13 in a vertical direction. The ionization unit 13 shown in FIG. 6 may allow for air to flow towards an inside of the ionization unit 13 from lateral directions through side openings 29 and may allow the air to flow out of the ionization unit 13 through an opening 31 at a lower side of the ionization unit 13.

In the illustrated embodiments, the ionization unit 13 moves at the ceiling 3 of the room 1. Thus, the ceiling 3 or parts thereof, such as the rails 15, form a support structure along which the ionization unit 13 moves. However, the ionization unit 13 could also move along a support structure separate from the ceiling 3.

Operation of the ionization unit 13 may be controlled by a control unit 33. In FIGS. 2, 4 and 6, the control unit 33 is shown at the ionization unit 13. However, it would be conceivable to provide the control unit 33 or parts of the control unit 33 external to the ionization unit 13. In this case, the control unit 33 or parts of the control unit 33 could be in communication with the ionization unit 13, in particular by wireless communication. The control unit 33 may control the ionizer 25 and the drive function of the ionization unit 13.

The ionization unit 13 may comprise one or more sensors 35. The one or more sensors 35 may, for example, comprise one or more of a moisture sensor determining moisture of the air, a particle sensor determining a particle density in the air and an obstacle sensor. The control unit 33 may control the ionization unit 13 to move along the ceiling 3 based on the output of one or more sensors 35. For example, the ionization unit 13 may be controlled to remain at its present location until a particle density in the air detected by the particle sensor is below a predetermined threshold. If the detected particle density is below the predetermined threshold, the control unit 33 may control the ionization unit 13 to move to another position.

In the embodiments of FIGS. 3 and 5, the ionization unit 13 moves freely at the ceiling 3. The control unit 33 may determine a path for movement of the ionization unit 13 based on pre-stored data. In addition or as an alternative, the control unit 33 may determine the path for moving the ionization unit 13 based on output of the obstacle sensor and a way-finding algorithm. The way-finding algorithm may store data from previous runs and may be self-improving.

The control unit 33 may be provided with information on activities carried out in the room 1 or sense information on activities carried out in the room. Based on the information on the activities, the control unit 33 could appropriately operate the ionization unit 13. The ionization unit 13 could be configured to move to a position at which an activity is carried out in the room 1. For example, the ionization unit 13 could be configured to follow movements of a person within the room 1.

Preferably, there is a cleaning robot 37 moving in coordination with the ionization unit 13. In FIGS. 1 and 3, the cleaning robot 37 moves on top of the floor 5. In FIG. 5, the cleaning robot 37 moves below the floor 5. The cleaning robot 37 in FIG. 1 may be the same as the cleaning robot 37 in FIG. 3. The cleaning robot 37 in FIG. 5 may have a different configuration. FIG. 7 shows details of the cleaning robot 37 of FIGS. 1 and 3. FIG. 8 shows details of the cleaning robot 37 of FIG. 5. Part A of FIG. 7 shows a top view of the cleaning robot 37 of FIGS. 1 and 3, Part B of the FIG. 7 shows a bottom view of the cleaning robot 37 of FIGS. 1 and 3 and Part C of FIG. 7 shows a top perspective view of the cleaning robot 37 of FIGS. 1 and 3. Part A of FIG. 8 shows a top view of the cleaning robot 37 of FIG. 5, Part B of the FIG. 8 shows a bottom view of the cleaning robot 37 of FIG. 5 and Part C of FIG. 8 shows a top perspective view of the cleaning robot 37 of FIG. 5.

The cleaning robot 37 comprises a robot body 39 and a drive unit for moving the cleaning robot 37 on a ground surface. The drive unit may comprise wheels 41. The wheels 41 may engage with the floor 5 or with a ground surface below the floor 5. An air inlet 43 for letting air into the robot body 39 is provided at the robot body 39. The air inlet 43 faces towards an upward direction. Further, an air outlet 45 is provided at the robot body 39. The cleaning robot 37 comprises an airflow unit configured to suck in air into the robot body 39 through the air inlet 43 and to discharge the air from the robot body 39 through the air outlet 45. An air passage connects the air inlet 43 with the air outlet 45 within the robot body 39.

At the air inlet 43, an air-permeable conductor 47 is provided. The air-permeable conductor 47 comprises and electrically charged surface configured to attract particles in the air that have been electrically charged by the ionization unit 13.

The cleaning robot 37 sucks in air including particles within the air from the room 1 and discharges the air and the particles through the outlet opening 45. The air may pass through the cleaning robot 37 without filtration. However, in principle, it would also be possible to provide a filter within the cleaning robot 37 to filter particles out of the air passing through the cleaning robot 37.

According to the embodiment shown in FIG. 7, the outlet opening 45 is provided at a bottom side of the cleaning robot 37. The outlet opening 45 is positioned at the robot body 39 such that air discharged through the air outlet 45 is directed towards the floor 5. The air discharged from the cleaning robot 37 may pass through the floor 5 into the circulation space 7.

In the embodiment shown in FIG. 8, which refers to the cleaning robot 37 moving below the floor 5 of the room 1, the air outlet 45 is laterally provided at the robot body 39. This leads to the air being laterally discharged within the circulation space 7.

The cleaning robot 37 comprises a control unit 49 controlling operation of the cleaning robot 37. The control unit 49 may control operation of the airflow unit and operation of the drive unit of the cleaning robot 37. Preferably, the cleaning robot 37 is controlled to move in coordination with ionization unit 13. The control unit 49 of the cleaning robot 37 may be in communication with the control unit 33 of the ionization unit 13 or with an external control unit to coordinate movement of the ionization unit 13 and the cleaning robot 37. The communication may be wireless communication.

In the coordinated movement of the ionization unit 13 and the cleaning robot 37, one of the ionization unit 13 and the cleaning robot 37 may be the lead unit and the other one may follow the lead unit. For example, the cleaning robot 37 may move according to a movement of the ionization unit 13.

The ionization unit 13 and the cleaning robot 37 may move so as to be positioned above each other. The cleaning robot 37 may move so as to be positioned below the ionization unit 13. According to an embodiment, the ionization unit 13 and the cleaning robot 37 may move directly above each other. Alternatively, the ionization unit 13 and the cleaning robot 37 may be positioned above each other within a certain tolerance. For example, the cleaning robot 37 may move on the floor 5 or below the floor 5 within a region around a vertical projection of the ionization unit 13 onto the floor 5. A distance between the cleaning robot 37 and the vertical projection of the ionization unit 13 onto the floor 5 may, for example, be kept lower than 5 m, or lower than 3 m, or lower than 2 m, or lower than 1 m, or lower than 0.5 m, or lower than 0.2 m, or lower than 0.1 m.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±10% of A.

1. Method for cleaning air in a room with a ceiling and a floor, comprising:
moving an ionization unit above the floor along a support structure provided at a distance to the floor; and
electrically charging particles in the air by the ionization unit.

2. Method according to claim 1, further comprising moving a cleaning robot at the floor of the room in coordination with the movement of the ionization unit.

3. Method according to claim 2, wherein the cleaning robot attracts charged particles in the air with at least one electrically charged surface.

4. Method according to claim 2, wherein the cleaning robot sucks in air and discharges the air in a downward direction.

5. Method according to claim 4, wherein the cleaning robot sucks in the air through an inlet opening facing towards an upside direction.

6. Method according to claim 1, wherein the ionization unit moves at the ceiling of the room.

7. Method according to claim 1, wherein the ionization unit hangs from the ceiling while moving at the ceiling.

8. Method according to claim 1, wherein the ionization unit is held at the support structure with suction cups provided at the ionization unit.

9. Method according to claim 1, wherein the ionization unit moves on top of the support structure.

10. System, comprising:
a room with a floor and a ceiling, wherein the room comprises a support structure provided at a distance to the floor; and
an ionization unit configured to move above the floor along the support structure and electrically charge particles within the room.

11. System according to claim 10, wherein the ceiling comprises the support structure.

12. System according to claim 10, wherein the ionization unit comprises suction cups configured to hold the ionization unit at the support structure.

13. System according to claim 10, wherein the support structure comprises a rail system.

14. System according to claim 10, further comprising a cleaning robot configured to move in coordination with the movement of the ionization unit.

15. Use of an ionization unit moving within a room along a support structure provided at a distance to a floor of the room to accelerate gravitation-based descent of particles within the room.
